(19) United States
(12) Patent Application Publication
(10) Pub. No.: US 2024/0091656 A1
(43) Pub. Date: Mar. 21, 2024

(54) Therapeutic Interactive Doll System

(71) Applicant: Adrienne Mazzetti, Rocky Point, NC (US)

(72) Inventor: Adrienne Mazzetti, Rocky Point, NC (US)

(21) Appl. No.: 17/946,472

(22) Filed: Sep. 16, 2022

(51) Int. Cl. A63H 3/00; A61B 5/00; A61B 5/16; A63H 3/36

(52) U.S. Cl. CPC A63H 3/003; A61B 5/165; A61B 5/7275; A63H 3/36; A61B 2562/0219; A63H 2200/00

(57) ABSTRACT

A therapeutic interactive doll system may be provided utilizing a doll body having a head, a torso, two arms and two legs. Each of the head, the torso, the two arms and the two legs may have an exterior surface enclosing an interior cavity. Each exterior surface may have a closable opening formed therein allowing access to the interior cavity. The system may utilize a central control module disposed within the doll body and a plurality of simulation elements disposed within the doll body. At least some of the plurality of simulation elements may be in electronic communication with the central control module.

### BACKGROUND OF THE INVENTION

It is estimated that someone develops initial symptoms of dementia every three seconds across the world. In total, the worldwide dementia population is estimated to be well above 60 million. In less economically developed countries, dementia patients may not have access to an effective first line treatment regimens. In more economically developed countries, dementia patients may be directed towards pharmaceutical interventions that carry a high cost burden. Therefore, it would be advantageous to provide a cost-effective first line treatment that provides better patient outcomes relative pharmaceutical options.

People living with dementia are often severely affected by the development of behavioral and psychological symptoms which represent a burden for both the patient and caregiver in the treatment setting. Specifically, such symptoms may include agitation, elation, wandering, depression, delusions and hallucinations. Across the industry, more than 80% of patients living with dementia will experience the development of these symptoms which often will cause the patient to be institutionalized.

While the use of psychotropic pharmaceuticals to treat these symptoms is ubiquitous across the dementia treatment industry, such drugs often have side effects that are not advantageous and may circuitously cause the symptoms to worsen over time. Specifically, the use of multiple psychotropic drugs to treat dementia patients has been shown to increase patient mortality while reducing patient physical and cognitive performance. Therefore, it would be advantageous to provide a first line non-pharmacologic approach for caregivers in treating dementia patients.

Amongst many non-pharmacologic interventions, doll therapy may be utilized as a useful tool to reduce behavioral and psychological symptoms in many nursing home patients affected by moderate to severe dementia. Doll therapy may be useful in reducing agitation and psychotropic drug administration while increasing patient quality of life. Further, doll therapy may also increase communication between the patient and caregiver as the doll stimulates conversation pertaining to parenting and caregiving as previously experienced in the patient's earlier life. However, one issue with doll therapy may be the extended time that the caregiver must spend in order to initiate and maintain interaction between the patent and doll.

Use of doll therapy may positively affect the patient's heart rate, cortisol level, blood pressure and similar stress indicators. Positively affecting these parameters causes the patient to exhibit more interactive behaviors with the caregiver. However, the characteristics of the doll used in therapy may directly affect the degree of success of the doll therapy. Generally, the more similar the doll can replicate interaction with a real human child, the higher the success rate of the doll therapy. Specifically, the more lifelike and interactive the doll's structural and functional features are designed, the more interactive the dementia patient becomes throughout the duration of the doll therapy.

Therefore, it would be advantageous to provide a therapeutic interactive doll system that increases overall verbal and physical interaction between a dementia patient, a doll and a caregiver. Further, it would be advantageous to provide a therapeutic interactive doll system that reduces a dementia patient's stress levels, depression symptoms, attention deficit and the like. Moreover, it would be advantageous to provide a therapeutic interactive doll system having the ability to accurately simulate the structural and functional characteristics of a human child. Additionally, it would be advantageous to provide a therapeutic interactive doll system that may be remotely controlled by the caregiver in order to automate the interaction between the patient and doll which increases the effectiveness of the caregiving while reducing its cost.

### BRIEF DESCRIPTION OF THE DRAWINGS

FIG. 1 is an overview of a therapeutic interactive doll system having a doll body in accordance with some embodiments of the present invention.

FIG. 2A is a detailed view of a foot of a doll body of a therapeutic interactive doll system in accordance with some embodiments of the present invention.

FIG. 2B is a detailed view of a hand of a doll body of a therapeutic interactive doll system in accordance with some embodiments of the present invention.

FIG. 2C is a detailed view of a torso of a doll body of a therapeutic interactive doll system in accordance with some embodiments of the present invention.

FIG. 2D is a detailed view of a head of a doll body of a therapeutic interactive doll system in accordance with some embodiments of the present invention.

FIG. 3 is a detailed view of a control module of a therapeutic interactive doll system in accordance with some embodiments of the present invention.

FIG. 4 is a detailed view of a remote simulation module of a therapeutic interactive doll system in accordance with some embodiments of the present invention.

### DETAILED DESCRIPTION OF THE INVENTION

Before describing the present invention in detail, it is to be understood that the invention is not limited to any one of the particular embodiments, which of course may vary. It is also to be understood that the terminology used herein is for the purpose of describing particular embodiments only, and therefore is not necessarily intended to be limiting. As used in this specification and the appended claims, terms in the singular and the singular forms “a,” “an,” and “the” include plural referents unless the context clearly dictates otherwise. Thus, for example, reference to “a therapeutic interactive doll system” also includes a plurality of therapeutic interactive doll systems and the like.

In some embodiments, a therapeutic interactive doll system is provided, comprising a doll body having a head, a torso, two arms and two legs, wherein: each of the head, the torso, the two arms and the two legs comprise an exterior surface enclosing an interior cavity, and each exterior surface comprises a closable opening formed therein allowing access to the interior cavity; a central control module disposed within the doll body; and a plurality of simulation elements disposed within the doll body, wherein at least some of the plurality of simulation elements are in electronic communication with the central control module.

In some embodiments, each closable opening comprises an open state and a closed state.

In some embodiments, the central control module is disposed within the torso of the doll body via the closable opening in the open state formed within the exterior surface of the torso.

In some embodiments, the plurality of simulation elements comprise a microphone.

In some embodiments, the plurality of simulation elements comprise an accelerometer.

In some embodiments, the plurality of simulation elements comprise an audio speaker.

In some embodiments, the plurality of simulation elements comprise a heating element.

In some embodiments, the plurality of simulation elements comprise a capacitive touch sensor.

In some embodiments, the plurality of simulation elements comprise a tactile feedback element.

In some embodiments, the plurality of simulation elements comprise a motion sensor.

In some embodiments, the plurality of simulation elements comprise a distributed weight element.

In some embodiments, the head comprises an audio speaker, an accelerometer and a microphone.

In some embodiments, at least some of the plurality of simulation elements detect one or more external stimuli events.

In some embodiments, responsive to detecting each external stimulus event, the plurality of simulation elements generate a detection signal that is communicated to the central control module.

In some embodiments, responsive to receiving the detection signal, the central control module generates one or more control signals that are communicated to one or more of the plurality of simulation elements.

In some embodiments, the plurality of simulation elements comprise an audio speaker; and responsive to the detecting the one or more external stimuli events, the audio speaker outputs one or more simulated baby noise audio segments.

In some embodiments, the plurality of simulation elements comprise a tactile feedback element; and responsive to detecting the one or more external stimuli events, the tactile feedback element generates one or more tactile actuation movements.

In some embodiments, a first portion of the doll body comprises a tactile feedback element and a capacitive touch sensor; and responsive to detecting one or more external stimuli events at the first portion of the doll body, the tactile feedback element generates one or more tactile actuation movements at the first portion of the doll body.

In some embodiments, a therapeutic interactive doll system is provided comprising a doll body having a head, a torso, two arms and two legs, wherein: each of the head, the torso, the two arms and the two legs comprise an exterior surface enclosing an interior cavity, and each exterior surface comprises a closable opening formed therein allowing access to the interior cavity; a central control module disposed within the doll body; and a plurality of sets of simulation elements comprising a plurality of simulation elements, wherein: each of the head, the torso, the two arms and the two legs comprise a set of simulation elements, at least some of the plurality of simulation elements are in electronic communication with the central control module, and responsive to detecting one or more external stimuli events, at least one of the plurality of simulation elements generate a detection signal that is communicated to the central control module.

In some embodiments, a therapeutic interactive doll system is provided comprising a doll body having a head, a torso, two arms and two legs, wherein: each of the head, the torso, the two arms and the two legs comprise an exterior surface enclosing an interior cavity, each exterior surface comprises a closable opening formed therein allowing access to the interior cavity, and each closable opening comprises an open state and a closed state; a central control module disposed within the torso of the doll body via the closable opening in the open state formed within the exterior surface of the torso; and a plurality of sets of simulation elements comprising a plurality of simulation elements, wherein: each of the head, the torso, the two arms and the two legs comprise a set of simulation elements, at least some of the plurality of simulation elements are in electronic communication with the central control module, responsive to detecting one or more external stimuli events, a first simulation element of the plurality of simulation elements generates a detection signal that is communicated to the central control module, and responsive to receiving the detection signal, the central control module generates one or more control signals that are communicated to at least a second simulation element of the plurality of simulation elements

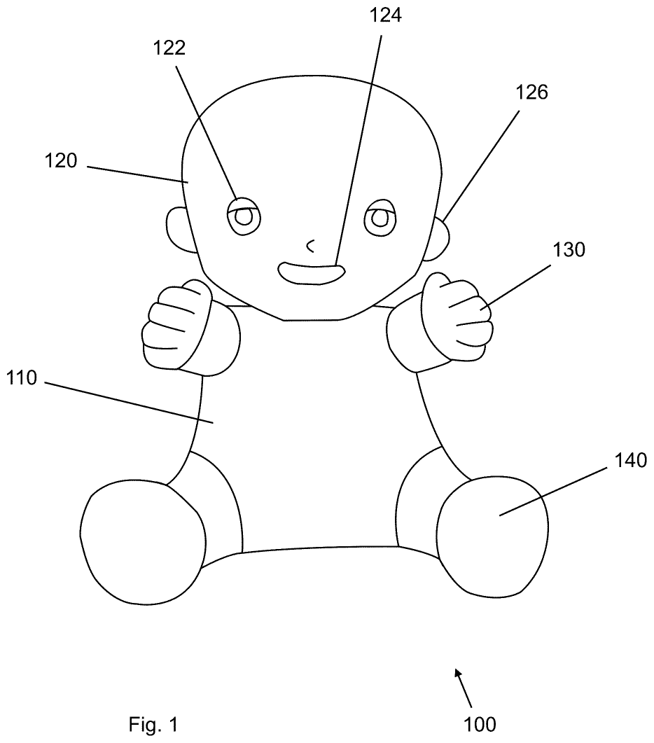

Exemplary embodiments of the present invention are illustrated in the accompanying figures. As shown in FIG. 1, overview of a therapeutic interactive doll system 100 having a doll body is provided. The doll body may comprise a torso 110, a head 120, two arms 130 and two legs 140. The head 120 may further comprise two eyes 122, a mouth 124 and two ears 126. Further, the two arms 130 may each comprise a hand and fiver fingers while the two legs 140 may each comprise a foot shape at the terminal end thereof.

While the doll body is shown in FIG. 1 to have a specific aesthetic form, it is contemplated that each of the torso 110, head 120, arms 130 and legs 140 may be structured and designed in any suitable manner so as to make the doll body resemble a real human child as closely as possible. For example, the head 120 may comprise hair disposed on the scalp thereof, the eyes 122 may be arranged closer together, the mouth 124 may comprise an opening, the legs 140 may comprise toes arranged on the feet thereof and the like.

Moreover, the doll body may be structured and designed in order to improve social interaction between a cognitively-impaired patient, an associated caregiver and the doll body. Specifically, the doll body may function in such a manner so as to replicate as closely as possible the bodily movements and audible sounds produced by a real human child. For instance, each of the torso 110, head 120, arms 130 and legs 140 may comprise distributed weighting elements that allow for the doll body to have a weight distribution similar to that of a real human child when held in the cognitively-impaired patient's arms. Further, each of the torso 110, head 120, arms 130 and legs 140 may comprise one or more thermal regulation elements that provide distributed warmth to the doll body similar to that of a real human child when touched by the patient.

Additionally, each of the torso 110, head 120, arms 130 and legs 140 may comprise one or more tactile feedback elements that allow for the doll body to move in manner akin to that of a real human child. Similarly, each of the torso 110, head 120, arms 130 and legs 140 may comprise one or more capacitive touch sensors that allow the doll body to sense when it is being touched by the cognitively-impaired patient so that it may respond accordingly and increase the social interaction between the patient and the doll body. Further, each of the torso 110, head 120, arms 130 and legs 140 may comprise one or more control units that serve to control the various functionalities of the doll body disclosed in this paragraph and the preceding paragraph.

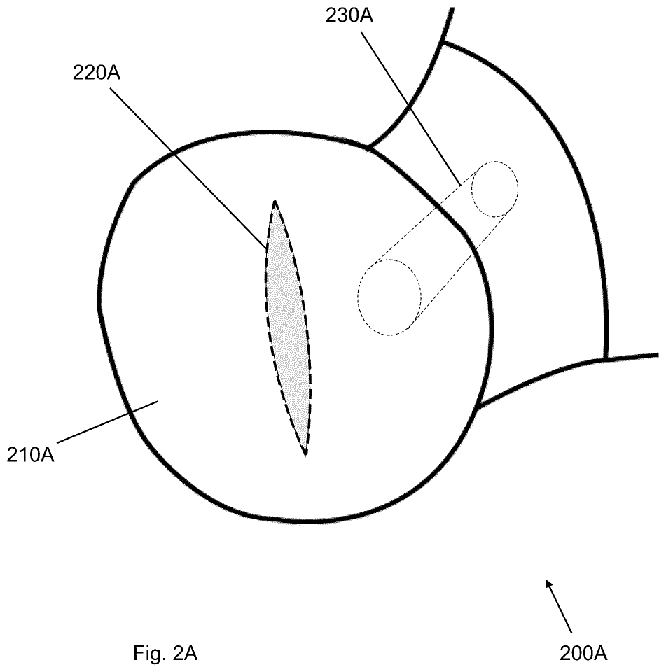

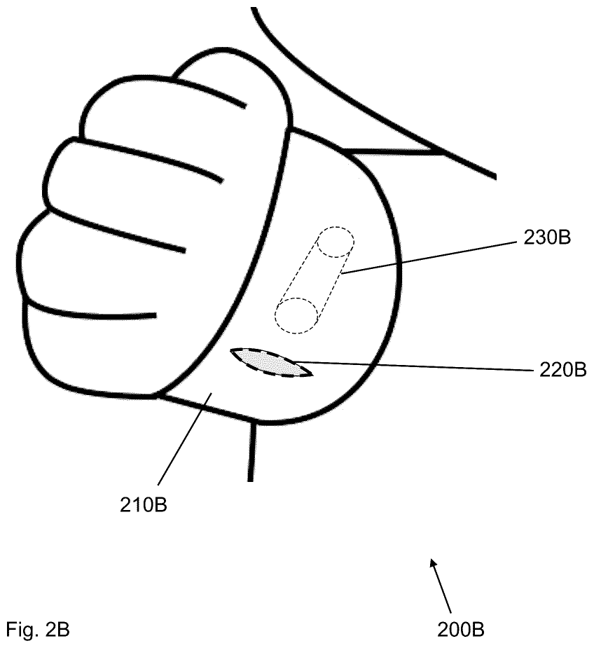

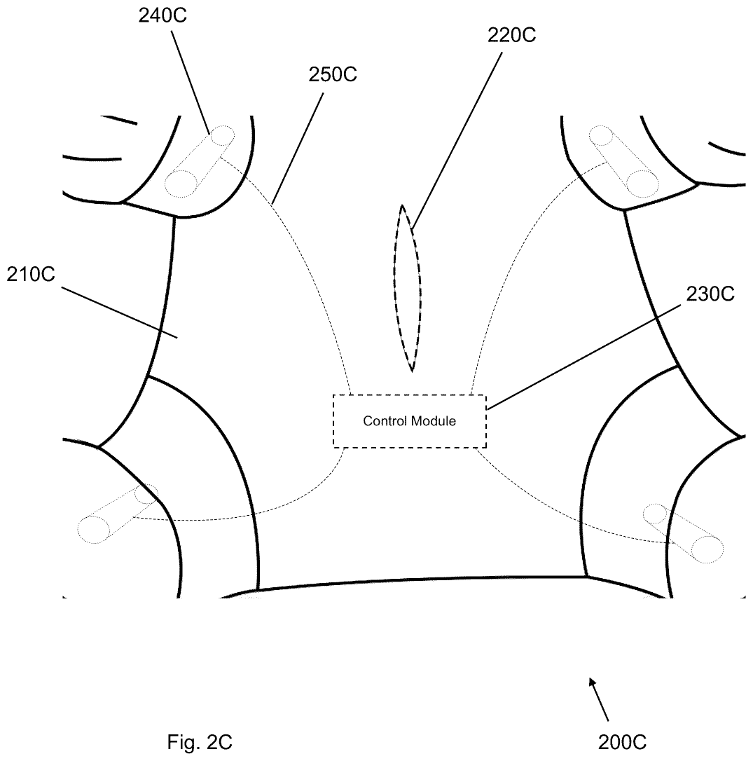

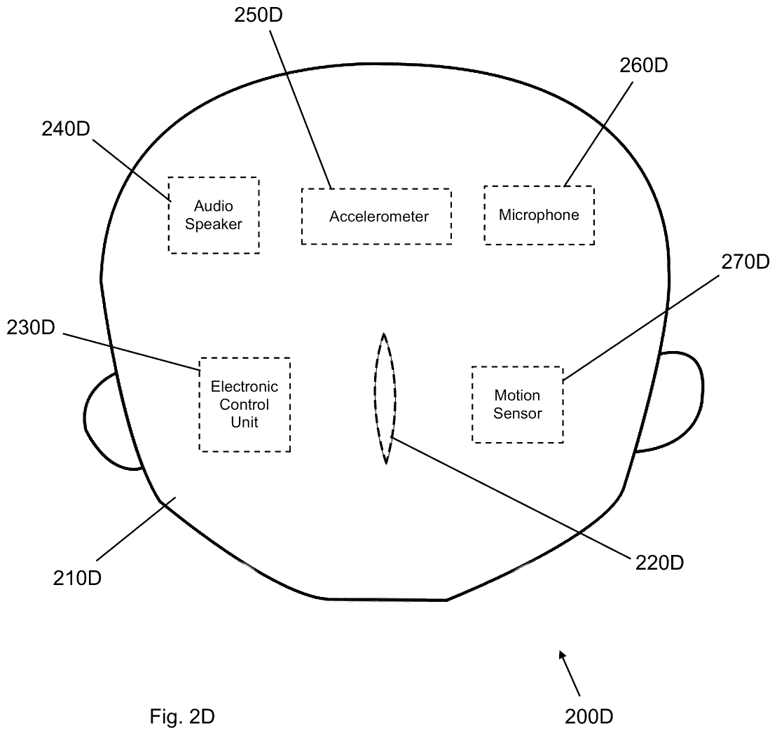

As shown in FIG. 2A, a detailed view of a leg 210A of a doll body 200A of a therapeutic interactive doll system is provided. The leg 210A may include a foot portion disposed at a terminal end thereof. The foot portion may comprise a closable opening 220A that allows a user to access the interior of the doll body 200A. Further, the closable opening 220A may also allow the user to selectively open and close the closable opening 220A to allow the user to insert and remove any desired structural elements. For example, the user may selectively insert one or more remote simulation modules 230A within the interior of the doll body 200A via the closable opening 220A and then close the opening 220A. Similarly, the user may selectively remove one or more remote simulation modules 230A from the interior of the doll body 200A via the closable opening 220A and then close the opening 220A.

The closable opening 220A is illustrated in FIG. 2A as being in the open state exposing the interior of the doll body 200A. The remote simulation module 230A is illustrated in FIG. 2A as having been inserted through the aperture of the closable opening 220A in the open state. While the remote simulation module 230A is illustrated as being cylindrical in shape, the remote simulation module 230A may be any suitable shape but is preferably elongate in nature so as to conform to the shape of the leg 210A and thereby better distribute heat, weight, tactile movement and the like throughout the given portion of the doll body 200A in which the remote simulation module 230A is situated.

As shown in FIG. 2B, a detailed view of an arm 210B of a doll body 200B of a therapeutic interactive doll system is provided. The arm 210B may include a hand portion disposed at a terminal end thereof. The hand portion may comprise a closable opening 220B that allows a user to access the interior of the doll body 200B. Further, the closable opening 220B may also allow the user to selectively open and close the closable opening 220B to allow the user to insert and remove any desired structural elements. For example, the user may selectively insert one or more remote simulation modules 230B within the interior of the doll body 200B via the closable opening 220B and then close the opening 220B. Similarly, the user may selectively remove one or more remote simulation modules 230B from the interior of the doll body 200B via the closable opening 220B and then close the opening 220B.

While the remote simulation module 230B is illustrated as being physically decoupled from a central control unit, the remote simulation module 230B may be directly coupled via wired circuitry to a central control unit or may be wirelessly coupled to the central control unit. Alternatively, each remote simulation module 230B may comprise a local control unit that is in communication with a central control unit which may be disposed within the doll body 200B or external thereto.

As shown in FIG. 2C, a detailed view of a torso 210C of a doll body 200C of a therapeutic interactive doll system is provided. The torso 210C may comprise a closable opening 220C that allows a user to access the interior of the doll body 200C. The torso 210C may couple together the arms and the legs of the doll body 200C such that a central control module 230C may act as a controller of functionality for each of the remote simulation modules 240C disposed in the various arms and legs of the doll body 200C. While the central control module 230C is illustrated as being coupled via wired circuitry 250C to the various remote simulation modules 240C, the central control module 230C may alternatively be wirelessly coupled to the remote simulation modules 240C via Bluetooth or other close-proximity communication protocol.

The central control module 230C may serve to control the functionality of the various remote simulation modules 240C based upon preprogrammed instructions and/or external input by a caregiver. For instance, the central control module 230C may control the heat distribution, tactile movement and the like of the doll body 200C via the remote simulation modules 240C. Similarly, the central control module 230C may process any inputs received by capacitive touch sensors, motion sensors, microphones and the like of the doll body 200C. While the torso 210C shown in FIG. 2 does not comprise a remote simulation module 240C therein, it may be preferable to provide one or more remote simulation modules 240C within the interior of the torso 210C via the closable opening 220C in order to provide a more lifelike doll body 200C that is akin to a real human child.

As shown in FIG. 2D, a detailed view of a head 210D of a doll body 200D of a therapeutic interactive doll system is provided. The head 210D may comprise a closable opening 220D that allows a user to access the interior of the doll body 200D. Further, the head 210D may comprise various electronic components disposed therein including, but not limited to, an electronic control unit 230D, an audio speaker 240D, an accelerometer 250D, a microphone 260D, a motion sensor 270D and the like. The electronic control unit 230D may control the functionality of the other electronic components via control signals received from a central control module or directly from a caregiver.

The audio speaker 240D may be utilized to transmit audio signals from the head 210D that are designed to increase the duration and quality of social interaction between the cognitively-impaired patient and the doll body 200D. Audio interaction is a fundamental aspect of social interaction and therefore is preferable to utilize during doll therapy with the patient. The accelerometer 250D may be utilized to measure various forces incident upon the head 210D and the doll body 200D more generally. By measuring force applied to the doll body 200D, the therapeutic interactive doll system may more accurately replicate interaction between the patient and a real human child. For instance, if a real human child is dropped, then the child will typically begin to cry. Therefore, if the doll body 200D is dropped, then the accelerometer 250D may measure that drop via force impact and transmit that information to the electronic control unit 230D which may then initiate a crying audio signal to be transmitted from the audio speaker 240D as a realistic response to the doll body 200D being dropped.

The microphone 260D may be utilized to record audio signals incident thereupon and transmit them to the electronic control unit 230D for processing and analysis. It would be advantageous to record and analyze the speech of the cognitively-impaired patient directed toward the doll body 200D as this will allow the therapeutic interactive doll system to interact with the speech of the patient in a more realistic manner akin to that of a real human child. The motion sensor 270D may be utilized to detect the proximity of a cognitively-impaired patient relative the doll body 200D. It would be advantageous to detect the patient's presence in order to activate the other electronic components out of a power-saving mode. Further, detecting the patient's presence allows the doll body 200D to initiate interaction with the patient via transmission of audio signals from the audio speaker 240D and via movement of legs and arms of the doll body 200D using one or more tactile feedback elements.

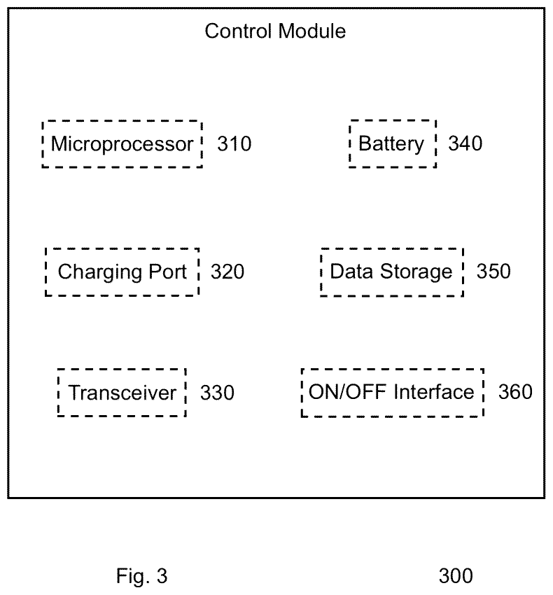

As shown in FIG. 3, a detailed view of a control module 300 of a therapeutic interactive doll system is provided. The control module 300 may be physically disposed within any portion of a doll body of the therapeutic interactive doll system including, but not limited to, the doll body's legs, arms, torso, head and the like. Further, the control module 300 may control the functionality of all other electronic modules contained within the doll body of the therapeutic interactive doll system.

In some embodiments, the control module 300 may comprise a microprocessor 310, a charging port 320, a transceiver 330, a battery 340, a data storage element 350, an ON/OFF interface 360 and the like or any combination thereof. The microprocessor 310 may control electronic signals sent between the other electronic components of the control module 300. For instance, the microprocessor 310 may execute preprogrammed instructions stored in the data storage element 350 to control power being input at the charging port 320 and passed on to the battery 340. Similarly, the microprocessor 310 may control communication signals from the transceiver 330 to one or more remote devices and may analyze communication signals to the transceiver 330 from one or more remote devices. Further, the microprocessor 310 may control the flow of power from the battery 340 to the other components based upon the biasing of the ON/OFF interface 360.

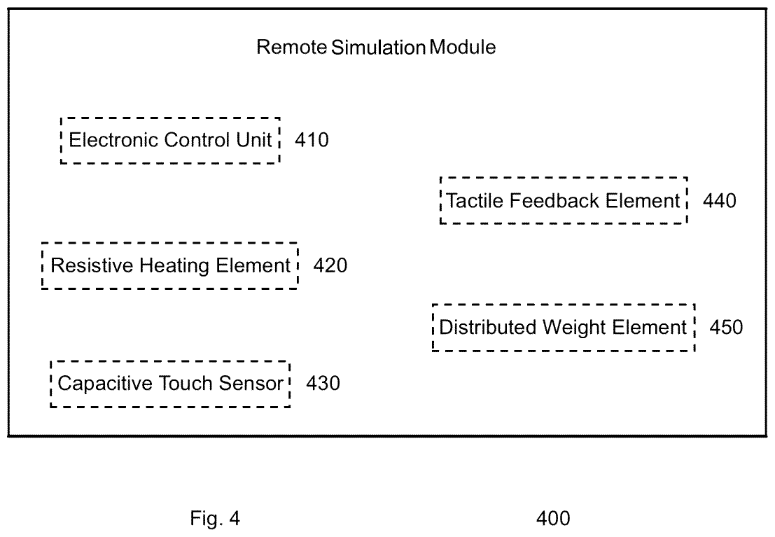

As shown in FIG. 4, a detailed view of a remote simulation module 400 of a therapeutic interactive doll system is provided. The remote simulation module 400 may be physically disposed in any portion of a doll body of the therapeutic interactive doll system such as, but not limited to, the doll body's legs, arms, torso, head and the like. Further, one or more remote simulation modules 400 may be utilized to animate the entirety of the doll body such that it might resemble that of a real human child when interacting with a cognitively-impaired patient.

In some embodiments, the remote simulation module 400 may comprise an electronic control unit 410, a resistive heating element 420, a capacitive touch sensor 430, a tactile feedback element 440, a distributed weight element 450 and the like or any combination thereof. The electronic control unit 410 may control the functionality of the other electronic components such as the resistive heating element 420, the capacitive touch sensor 430 and the tactile feedback element 440. Further, the electronic control unit 410 may receive electronic control signals from another central control module or directly from a control source external to the doll body such as a patient caregiver.

The resistive heating element 420 may allow supplied electric power to be transformed via one or more resistors into thermal energy to be applied to the doll body in order to provide realistic sense of warmth thereto when handled by a cognitively-impaired patient. The capacitive touch sensor 430 may detect the touch of the patient and transmit a signal of that detection to the electronic control unit 410 for further action to be taken in response to such detection. For instance, the electronic control unit 410 may actuate the tactile feedback element 440 to provide local movement in response to detecting the patient's touch in that local area of the doll body.

The tactile feedback element 440 may also be used to initiate interaction with the cognitively-impaired patient by moving the doll body in a manner simulating that of a real human child to illicit responses from the patient. Further, the distributed weight element 450 may provide an amount of weight to the doll body in a manner that simulates the weight feeling of a real human child when held by the patient. To this end, it would be advantageous to provide an elongate shape to the distributed weight element 450 so that the weight may be evenly distributed throughout the extent of the various portions of the doll body similar to that of a real human child.

With reference to FIGS. 1-4, the disclosed electronic control unit, resistive heating elements, capacitive touch sensors, tactile feedback elements, distributed weight elements, audio speakers, accelerometers, microphones, motion sensors, control modules may collectively be referred to as simulation elements given each of these elements allow the doll body of the therapeutic interactive doll system to better simulate the characteristics of a human baby.

With reference to FIGS. 1-4, the therapeutic interactive doll system may utilize a transceiver that communicates over a wireless protocol, such as Bluetooth communication protocol, with a remote device programmed to communicate therewith. The remote device may take the form of an existing smartphone, smartwatch, desktop computer, tablet or like device. Alternatively, the remote device may take the form of a uniquely fabricated remote communication device that is specifically made for the purposes of communicating with the transceiver of the therapeutic interactive doll system.

With reference to FIGS. 1-4, the therapeutic interactive doll system may utilize the remote device to control various functionality of the doll body. For instance, the remote device may communicate control signals to the transceiver within the doll body to execute movements of various parts of the doll body. Specifically, the remote device may control the eyelids of the doll body such that the eyelids may be manipulated between open and closed states based upon control signals sent by the remote device and executed by one or more control modules within the doll body. The control module may control an electric stepper motor or the like which is coupled to the eyelids in order to manipulate the eyelids between the open and closed states.

Similarly, the remote device may communicate control signals to the transceiver within the doll body to control the torso of the doll body such that the chest of the torso may be manipulated between expanded and compressed states based upon the control signals sent by the remote device and executed by one or more control modules within the doll body. The control modules may control one or more tactile feedback elements which are disposed within the torso and adjacent the outer surface of the chest of the doll body in order to actuate the outer surface of the chest between the expanded and compressed states.

With reference to FIGS. 1-4, the tactile feedback device may take the form of any iterative actuation device utilizing an electric motor such as, but not limited to, an electric motor reciprocating an off-center weight, an electric stepper motor moving its housing between expanded and compressed states, an electric motor actuating an elongate joint between extended and retracted states, an electric motor manipulating air volume within a cavity between inflated and deflated states and the like or any combination thereof.

Similarly, the remote device may communicate control signals to the transceiver within the doll body to control the temperature of the outer surface of the doll body such that a simulated human body temperature may be achieved at the outer surface when it is felt by the patient's hand. The outer surface temperature may be regulated based upon the control signals sent by the remote device and executed by one or more control modules within the doll body. The control modules may control one or more resistive heating elements which are disposed throughout the doll body in order to provide a realistic sense of a simulated human body temperature to the outer surface of the doll body.

With reference to FIGS. 1-4, the doll body may comprise a thermally conductive material disposed adjacent to one or more portions of the outer surface of the doll body so that fewer resistive heating elements need to be utilized in order to spread thermal energy evenly across the doll body outer surface. In other embodiments, each resistive heating element may be fabricated as a metallic sheet or planar metallic wire configuration and disposed adjacent the outer surface of the doll body in order to spread thermal energy evenly thereacross. In any of these embodiments, the outer surface material of the doll body may be fabricated with thermally conductive material contained therein in order to further evenly distribute thermal energy thereacross. As such, the resistive heating elements may be disposed within the remote simulation modules, respectively, and/or may be disposed outside of the remote simulation modules.

Similarly, the remote device may communicate control signals to the transceiver within the doll body to control audio output from the doll body such that audio may be output from the audio speaker contained within the doll body based upon the control signals sent by the remote device and executed by one or more control modules within the doll body. The control modules may control the audio speaker to output audio as preprogrammed according to software stored within a memory device contained within the control modules or an electronic control unit. In this and other embodiments, a control module and an electronic control unit are synonymous terms utilized for the same or similar electronic component comprising a processor, non-transitory machine-readable memory containing software stored thereupon.

With reference to FIGS. 1-4, the audio output from the audio speakers of the doll body may output audio simulating that of typical noises created by a baby. Such simulated baby noise audio output may include, but is not limited to, talking sounds, sneezing sounds, gurgling sounds, hiccupping sounds, sleeping sounds, snorting sounds, nasal whistling sounds from breathing, inhalation and exhalation sounds, laughing sounds, whining sounds, crying sounds and the like or any combination thereof.

With reference to FIGS. 1-4, the remote device may control the audio output from the audio speakers of the doll body. Alternatively, the audio speakers may be preprogrammed to automatically generate audio output based upon detected external stimuli events without the need for manual control signals to be sent from the remote device. In other embodiments, both automatic and manual audio output may be generated by the audio speakers of the doll body. The detected external stimuli events may include, but are not limited to, motion external to the doll body detected by a motion sensor, forces incident upon the doll body detected by an accelerometer, audio signals not generated by the audio speakers and detected by a microphone, human touch contact with the doll body detected by a capacitive touch sensor and the like or any combination thereof. Upon detection of the one or more external stimuli, a detection signal may be generated and communicated to the one or more control modules which may then send a control signal to the audio speaker to output therefrom one or more simulated baby noise audio segments described above.

With reference to FIGS. 1-4, a simulated baby noise audio segment may be output from the audio speaker for each separate external stimuli detected. For instance, motion detected by the motion sensor may trigger the audio speaker to output one or more neutral mood simulated baby noise audio segments including, but not limited to, sleeping sounds, nasal whistling sounds from breathing, inhalation and exhalation sounds and the like or any combination thereof. Further, forces incident upon the doll body that exceed a predetermined force threshold as detected by the accelerometer may trigger the audio speaker to output one or more negative mood simulated baby noise audio segments such as whining sounds and crying sounds. Forces incident upon the doll body that do not exceed the predetermined force threshold may trigger the audio speaker to output one or more neutral or positive mood simulated baby noise audio segments such as talking sounds, sneezing sounds, gurgling sounds, hiccupping sounds, sleeping sounds, snorting sounds, nasal whistling sounds from breathing, inhalation and exhalation sounds, laughing sounds and the like or any combination thereof. Audio signals above a predetermined decibel threshold as detected by the microphone of the doll body may trigger the audio speaker to output one or more negative mood simulated baby noise audio segments such as whining sounds and crying sounds. Audio signals below a predetermined decibel threshold as detected by the microphone of the doll body may trigger the audio speaker to output one or more neutral or positive mood simulated baby noise audio segments such as talking sounds, sneezing sounds, gurgling sounds, hiccupping sounds, sleeping sounds, snorting sounds, nasal whistling sounds from breathing, inhalation and exhalation sounds, laughing sounds and the like or any combination thereof. Human touch contact detected by the capacitive touch sensor may trigger the audio speakers to output one or more neutral or positive mood simulated baby noise audio segments such as talking sounds, sneezing sounds, gurgling sounds, hiccupping sounds, sleeping sounds, snorting sounds, nasal whistling sounds from breathing, inhalation and exhalation sounds, laughing sounds and the like or any combination thereof.

In the event multiple external stimuli are detected by the doll body within a short period of time, those stimuli triggering negative mood simulated baby noise audio segments will take priority over the other detected stimuli. Specifically, any stimuli triggering negative mood simulated baby noise audio segments may prevent the doll body from triggering neutral or positive mood simulated baby noise audio segments for a predetermined period of time in order to more closely simulate the bodily responses of a human baby. In the event multiple external stimuli are detected that each trigger neutral or positive mood simulated baby noise audio segments, the control module will interpret these as a single external stimuli detected and will commence output of neutral or positive mood simulated baby noise audio segments. Similarly, in the event multiple external stimuli are detected that each trigger negative mood simulated baby noise audio segments, the control module will interpret these as a single external stimuli detected and will commence output of negative mood simulated baby noise audio segments.

With reference to FIGS. 1-4, the actuation of the one or more tactile feedback elements may be triggered upon detection of the external stimuli by the doll body. For instance, triggering of neutral or positive mood simulated baby noise audio segments may simultaneously trigger actuation of the one or more tactile feedback elements. Similarly, triggering of negative mood simulated baby noise audio segments may simultaneously trigger actuation of the one or more tactile feedback elements. Further, the rate at which the one or more tactile feedback elements are actuated may vary upon which simulated baby noise audio segment is being output by the audio speakers of the doll body. For example, crying sounds being output may increase the rate at which the one or more tactile feedback elements are being actuated whereas sleeping sounds being output may decrease the rate at which the one or more tactile feedback elements are being actuated. Additionally, the one or more tactile feedback elements may be actuated responsive to one or more of the capacitive touch sensors sensing touch. Specifically, a tactile feedback element may be triggered to generate movement in a given portion of the doll body in response to a capacitive touch sensor sensing touch in the given portion of the doll body. This further provides a more realistic simulation of the touch-responsiveness characteristics found in a human baby.

With reference to FIGS. 1-4, the simulation elements such as the electronic control unit, resistive heating elements, capacitive touch sensors, tactile feedback elements, distributed weight elements, audio speakers, accelerometers, microphones, motion sensors, control modules and the like that are illustrated in FIGS. 1-4 as being contained within a remote simulation module may not be contained therein but, rather, may be disposed on their own in an individual manner within the interior of the doll body. In some embodiments, one or more sets of simulation elements may be distributed within one or more portions of the doll body such as the head, the torso, the arms, the legs and the like.

With reference to FIGS. 1-4, the remote device may communicate control signals to the transceiver within the doll body to control an overall patient sensitivity setting within the control unit in order to accommodate the mental state of the patient who is interacting with the doll body. For instance, the overall patient sensitivity setting may comprise a scale of values from the lowest sensitivity value to the highest sensitivity value. The sensitivity value may be selected based upon the mental state of the patient undergoing treatment.

Lower sensitivity settings may be utilized for patients who do not exhibit severe mental debilitation but rather are only at the early stages of mental decline and so can normally interact with the doll body. In such a situation, the patient may more attune to the synthetic nature of the doll body. Therefore, all simulated functionality of the doll body, including negative mood simulated baby noise audio segments, may be utilized in order to make the doll body appear as realistic as possible.

Higher sensitivity settings may be utilized for patients who exhibit severe mental debilitation such that their perception of reality is skewed and/or they become very easily agitated and sensitive to negative circumstances. In such a situation, the patient may regress in their treatment if they encounter negative mood simulated baby noise audio segments. Therefore, only neutral or positive mood simulated baby noise audio segments may be utilized in order to prevent the debilitated patient from enduring further mental duress due to the negative interaction.

The specification and drawings are to be regarded in an illustrative rather than a restrictive sense. However, it will be evident that various modifications and changes may be made thereunto without departing from the broader spirit and scope of the invention as set forth in the claims. Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

Throughout this disclosure, the phrase ‘modularly coupled’ and similar terms and phrases are intended to convey that any element of a given class of elements may be coupled to another given element and vice versa with equal effect. For example, any extension cord of a plurality of extension cords may be modularly coupled to another extension cord and vice versa with equal effect. Further, throughout this disclosure, the phrase ‘removably coupled’ and similar terms and phrases are intended to convey that a given element may be iteratively coupled to and removed from another given element as desired. For example, a male plug of a first extension cord may be removably coupled to a female plug of a second extension cord as desired.

The use of the terms “a,” “an,” “the,” and similar referents in the context of describing the disclosed embodiments (especially in the context of the following claims) are to be construed to cover both the singular and the plural, unless otherwise indicated or clearly contradicted by context. The terms “comprising,” “having,” “including,” and “containing” are to be construed as open-ended terms (i.e., meaning “including, but not limited to,”) unless otherwise noted. The term “connected,” where unmodified and referring to physical connections, is to be construed as partly or wholly contained within, attached to, or joined together, even if there is something intervening. Recitation of ranges of values are merely intended to serve as a shorthand method of referring individually to each separate value falling within the range, unless otherwise indicated and each separate value is incorporated into the specification as if it were individually recited. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

Conjunctive language, such as phrases of the form “at least one of A, B, and C,” or “at least one of A, B and C,” is understood with the context as used in general to present that an item, term, etc., may be either A or B or C, or any nonempty subset of the set of A and B and C, unless specifically stated otherwise or otherwise clearly contradicted by context. For instance, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present. In addition, unless otherwise noted or contradicted by context, the term “plurality” indicates a state of being plural (e.g., “a plurality of items” indicates multiple items). The number of items in a plurality is at least two, but can be more when so indicated either explicitly or by context.

The use of any examples, or exemplary language (e.g., “such as”) provided, is intended merely to better illuminate embodiments of the invention and does not pose a limitation on the scope of the invention unless otherwise claimed. No language in the specification should be construed as indicating any non-claimed element as essential to the practice of the invention.

Embodiments of this disclosure are described, including the best mode known to the inventors for carrying out the invention. Variations of those embodiments may become apparent to those of ordinary skill in the art upon reading the foregoing description. The inventors expect skilled artisans to employ such variations as appropriate and the inventors intend for embodiments of the present disclosure to be practiced otherwise than as specifically described. Accordingly, the scope of the present disclosure includes all modifications and equivalents of the subject matter recited in the claims appended hereto as permitted by applicable law. Moreover, although above-described elements may be described in the context of certain embodiments of the specification, unless stated otherwise or otherwise clear from context, these elements are not mutually exclusive to only those embodiments in which they are described; any combination of the above-described elements in all possible variations thereof is encompassed by the scope of the present disclosure unless otherwise indicated or otherwise clearly contradicted by context.

All references, including publications, patent applications, and patents, cited are hereby incorporated by reference to the same extent as if each reference were individually and specifically indicated to be incorporated by reference and were set forth in its entirety.

1. A therapeutic interactive doll system, comprising:
a doll body having a head, a torso, two arms and two legs, wherein:
each of the head, the torso, the two arms and the two legs comprise an exterior surface enclosing an interior cavity, and
each exterior surface comprises a closable opening formed therein allowing access to the interior cavity;
a central control module disposed within the doll body; and
a plurality of simulation elements disposed within the doll body, wherein at least some of the plurality of simulation elements are in electronic communication with the central control module.

2. The therapeutic interactive doll system of claim 1, wherein each closable opening comprises an open state and a closed state.

3. The therapeutic interactive doll system of claim 1, wherein the central control module is disposed within the torso of the doll body via the closable opening in the open state formed within the exterior surface of the torso.

4. The therapeutic interactive doll system of claim 1, wherein the plurality of simulation elements comprise a microphone.

5. The therapeutic interactive doll system of claim 1, wherein the plurality of simulation elements comprise an accelerometer.

6. The therapeutic interactive doll system of claim 1, wherein the plurality of simulation elements comprise an audio speaker.

7. The therapeutic interactive doll system of claim 1, wherein the plurality of simulation elements comprise a heating element.

8. The therapeutic interactive doll system of claim 1, wherein the plurality of simulation elements comprise a capacitive touch sensor.

9. The therapeutic interactive doll system of claim 1, wherein the plurality of simulation elements comprise a tactile feedback element.

10. The therapeutic interactive doll system of claim 1, wherein the plurality of simulation elements comprise a motion sensor.

11. The therapeutic interactive doll system of claim 1, wherein the plurality of simulation elements comprise a distributed weight element.

12. The therapeutic interactive doll system of claim 1, wherein the head comprises an audio speaker, an accelerometer and a microphone.

13. The therapeutic interactive doll system of claim 1, wherein at least some of the plurality of simulation elements detect one or more external stimuli events.

14. The therapeutic interactive doll system of claim 13, wherein, responsive to detecting each external stimulus event, the plurality of simulation elements generate a detection signal that is communicated to the central control module.

15. The therapeutic interactive doll system of claim 14, wherein, responsive to receiving the detection signal, the central control module generates one or more control signals that are communicated to one or more of the plurality of simulation elements.

16. The therapeutic interactive doll system of claim 13, wherein:
the plurality of simulation elements comprise an audio speaker; and
responsive to the detecting the one or more external stimuli events, the audio speaker outputs one or more simulated baby noise audio segments.

17. The therapeutic interactive doll system of claim 13, wherein:
the plurality of simulation elements comprise a tactile feedback element; and
responsive to detecting the one or more external stimuli events, the tactile feedback element generates one or more tactile actuation movements.

18. The therapeutic interactive doll system of claim 1, wherein:
a first portion of the doll body comprises a tactile feedback element and a capacitive touch sensor; and
responsive to detecting one or more external stimuli events at the first portion of the doll body, the tactile feedback element generates one or more tactile actuation movements at the first portion of the doll body.

19. A therapeutic interactive doll system, comprising:
a doll body having a head, a torso, two arms and two legs, wherein:
each of the head, the torso, the two arms and the two legs comprise an exterior surface enclosing an interior cavity, and
each exterior surface comprises a closable opening formed therein allowing access to the interior cavity;
a central control module disposed within the doll body; and
a plurality of sets of simulation elements comprising a plurality of simulation elements, wherein:
each of the head, the torso, the two arms and the two legs comprise a set of simulation elements,
at least some of the plurality of simulation elements are in electronic communication with the central control module, and
responsive to detecting one or more external stimuli events, at least one of the plurality of simulation elements generate a detection signal that is communicated to the central control module.

20. A therapeutic interactive doll system, comprising:
a doll body having a head, a torso, two arms and two legs, wherein:
each of the head, the torso, the two arms and the two legs comprise an exterior surface enclosing an interior cavity,
each exterior surface comprises a closable opening formed therein allowing access to the interior cavity, and
each closable opening comprises an open state and a closed state;
a central control module disposed within the torso of the doll body via the closable opening in the open state formed within the exterior surface of the torso; and
a plurality of sets of simulation elements comprising a plurality of simulation elements, wherein:
each of the head, the torso, the two arms and the two legs comprise a set of simulation elements,
at least some of the plurality of simulation elements are in electronic communication with the central control module,
responsive to detecting one or more external stimuli events, a first simulation element of the plurality of simulation elements generates a detection signal that is communicated to the central control module, and
responsive to receiving the detection signal, the central control module generates one or more control signals that are communicated to at least a second simulation element of the plurality of simulation elements.
